(19) United States
(12) Patent Application Publication
(10) Pub. No.: US 2006/0069340 A1
(43) Pub. Date: Mar. 30, 2006

(54) Shunt for the treatment of glaucoma

(75) Inventor: Gabriel Simon, Madrid (ES)

Correspondence Address: MAINE & ASMUS, 100 MAIN STREET, P O BOX 3445, NASHUA, NH, 03061-3445, US

(73) Assignee: SOLX, INC., Boston, MA (US)

(21) Appl. No.: 11/266,055

(22) Filed: Nov. 3, 2005

Related U.S. Application Data
(63) Continuation-in-part of application No. 10/869,166, filed on Jun. 16, 2004.
(60) Provisional application No. 60/624,686, filed on Nov. 3, 2004.
(60) Provisional application No. 60/478,895, filed on Jun. 16, 2003.

(51) Int. Cl. A61M 5/00; A61F 2/14

(52) U.S. Cl. 604/8; 623/4.1

(57) ABSTRACT

A system is provided for reducing intraocular pressure, the system having: an implantable shunt, the implantable shunt with a planar member, at least one microchannel disposed within that planar member, and a laser whereby at least one fenestration may be introduced into the microchannel.

### RELATED APPLICATIONS

This application claims the benefit of U.S. Provisional Application No. 60/624,686, filed Nov. 3, 2004 and is a continuation in part of U.S. application Ser. No. 10/869166 filed Jun. 16, 2004 which in turn claims the benefit of U.S. Provisional Application No. 60/478,895, filed Jun. 16, 2003. These applications are herein incorporated in their entirety by reference.

### FIELD OF THE INVENTION

The present invention pertains to surgical treatments for glaucoma and methods for reducing intraocular pressure (IOP), and more particularly relates to an implantable shunt device for allowing aqueous outflows from the eye's anterior chamber and associated methods thereof.

### BACKGROUND OF THE INVENTION

Glaucoma is a major public health problem, affecting about two percent of the U.S. population and the third most common cause of blindness in the U.S. There are several forms of glaucoma however each results in elevated intraocular pressure (IOP) in the eye, which can cause progressive damage to the optic nerve, and both central and peripheral visual field loss. If the IOP remains high for an extended period of time, total vision loss can occur. The elevated IOP is caused by an imbalance in fluid inflows and outflows in the eye, and the pressure reduces the blood supply to the optic nerve. The principal objective of medical treatment is the lowering of intraocular pressure.

The anterior chamber of the eye contains the aqueous humor, a clear fluid that is produced continuously by the ciliary body around the lens. The constant flow of aqueous humor though the eye's front chamber exits through two different routes. A limited outflow occurs through the uveoscleral route, wherein fluid migrates outwardly between muscle fibers of the ciliary body. The primary aqueous outflow pathway is through the trabecular meshwork (TM) and the Schlemm's canal.

The trabecular meshwork is a filtering structure that extends around the circumference of the eye at the “angle”—the junction between the iris, sclera and cornea. The trabecular meshwork consists of layers of collagen webs that filter the outflows. The meshwork has a monolayer of trabecular cells that produce enzymes for degrading extracellular material that may be captured by the filtering structure.

Aqueous humor that passes through the trabecular meshwork flows into Schlemm's canal, which is a passageway or series of septae that extend around the circumference of the eye adjacent to the meshwork. The aqueous fluid thereafter flows through a series of collecting channels that drain from Schlemm's canal and into the episcleral venous system. In a normal eye, aqueous production by the ciliary body is equal to aqueous outflows to provide an IOP that remains constant in the 15 to 21 mm Hg range. In a patient suffering from glaucoma, the resistance through the outflow system is typically greater than 21 mm Hg. In primary open angle glaucoma (POAG), the most common form of glaucoma, the principal resistance to fluid outflow is centered about the region of trabecular meshwork that is adjacent Schlemm's canal. It is believed that an abnormal trabecular cell metabolism results in compacted meshwork layers or a build up of extracellular materials within the meshwork that inhibits fluid flows.

Numerous therapies have been developed for treating glaucoma by decreasing intraocular pressure. Pharmacological therapies include topical ophthalmic drops and oral medications that reduce the production of aqueous by the ciliary body or increase aqueous outflows via the uveoscleral route. The treatments generally require applications at least daily and are relatively expensive. Furthermore, drugs may have occasional serious side effects, such as blurred vision, allergic reactions, headaches and potentially dangerous interactions with other drugs.

Surgical approaches for treating open-angle glaucoma consist of laser trabeculoplasty, trabeculectomy, and the implantation of aqueous shunts. Trabeculectomy is a widely practiced surgery wherein microsurgical techniques are used to dissect the trabecular meshwork to allow more rapid aqueous outflow through the meshwork. The benefits of the dissection procedures diminish over time due to the body's wound healing response and resulting fibrosis that repairs and closes the dissected opening in the meshwork. After the dissections are healed up, the intraocular pressure again increases. Thus these expensive procedures do not provide a long-lasting cure.

Implantable shunts and surgical methods are also known for providing a fluid path for aqueous humor to exit the anterior chamber of the eye to the sclera or a space beneath the conjunctiva. See e.g., U.S. Pat. No. 6,050,970 to Baerveldt.

Trabeculectomies and shunt surgeries and variations thereof have several disadvantages and moderate success rates. Such surgeries require significant surgical skills to create an incision through the full thickness of the sclera into the subconjunctival space. Further, the surgeries cause substantial trauma to the eye. The procedures are generally performed in an operating room and have a prolonged recovery time. Thus, the state of the art shunts and surgical techniques have yet to provide a cost-effective and long-lasting solution which has short recovery periods and low risk.

What are needed, therefore, are devices and techniques for successful, long-term reduction in intraocular pressure.

### SUMMARY OF THE INVENTION

One embodiment of the present invention provides a system for reducing intraocular pressure, the system comprising: an implantable shunt, the implantable shunt comprising; a substantially planar member; at least one microchannel disposed within the planar member; and a laser whereby at least one fenestration may be introduced into the microchannel.

Another embodiment of the present invention provides such a system further comprising a closed cavity containing a pharmaceutical agent.

A further embodiment of the present invention provides such a system wherein the cavity is smaller than 1 mm in diameter.

A still further embodiment of the present invention provides such a system wherein the laser has a wavelength between about approximately 750 nm and about approximately 800 nm.

Still another embodiment of the present invention provides such a system wherein a pharmaceutical agent is applied as a coating to the implantable shunt.

An even further embodiment of the present invention provides such a system further comprising pre-implantation configured inlet and outlet apertures.

Yet another embodiment of the present invention provides such a system wherein the laser is a titanium sapphire laser.

A yet still further embodiment of the present invention provides such a system wherein the laser is configured to close the fenestrations when applied by a user to the edge of the fenestration.

One embodiment of the present invention provides a method for decreasing ocular hypertension, the method comprising: Implanting a microchannel shunt having at least one microchannel; and adjusting the flow of aqueous fluid through the shunt using a laser light beam having a wavelength of between about approximately 750 and about approximately 800 nm.

Another embodiment of the present invention provides such a method wherein the shunt comprises a pharmaceutical agent.

A further embodiment of the present invention provides such a method wherein the agent is selected from the group of agents consisting of beta blockers, alpha-2 antagonists, and prostaglandin analogues.

Still another embodiment of the present invention provides such a method further comprising exposing the agent to the aqueous fluid by laser manipulation of the shunt.

A still further embodiment of the present invention provides such a method wherein the step of adjusting the flow of aqueous fluid through the shunt comprises introducing at least one fenestration communicating with the microchannel.

Yet another embodiment of the present invention provides such a method wherein the step of adjusting the flow of aqueous fluid through the shunt comprises gradually closing a fenestration in the microchannel.

One embodiment of the present invention provides an apparatus for the treatment of ocular hypertension, the apparatus comprising: a microchannel shunt, the shunt having a microchannel, and being configured to receive a laser pulse having a wavelength of between 750 and 800 nm; fenestrations disposed within the microchannel, the fenestrations being configured to be opened and closed by operation of the laser.

Another embodiment of the present invention provides such an apparatus wherein the shunt contains a pharmaceutical agent.

A further embodiment of the present invention provides such an apparatus wherein the agent is selected from the group of agents consisting of beta blockers, alpha-2 antagonists, and prostaglandin analogues.

Still another embodiment of the present invention provides such an apparatus wherein the shunt is longer than 4 mm.

A still further embodiment of the present invention provides such an apparatus the shunt is between about approximately 5 mm and about approximately 10 mm in length.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a perspective view of a shunt configured in accordance with one embodiment of the present invention.

FIG. 2 is a perspective view of such a shunt configured in accordance with one embodiment of the present invention disposed in a patient's eye.

FIG. 3A is a cross sectional elevation view of a closed shunt fenestration configured in accordance with one embodiment of the present invention.

FIG. 3B is a cross sectional elevation view of an open shunt fenestration configured in accordance with one embodiment of the present invention.

FIG. 3C is a cross sectional elevation view of a partially closed shunt fenestration configured in accordance with one embodiment of the present invention.

FIG. 4 is a perspective view of a shunt configured in accordance with one embodiment of the present invention.

FIG. 5 is a perspective elevation view of an elongate shunt configured in accordance with one embodiment of the present invention.

### DETAILED DESCRIPTION OF THE INVENTION

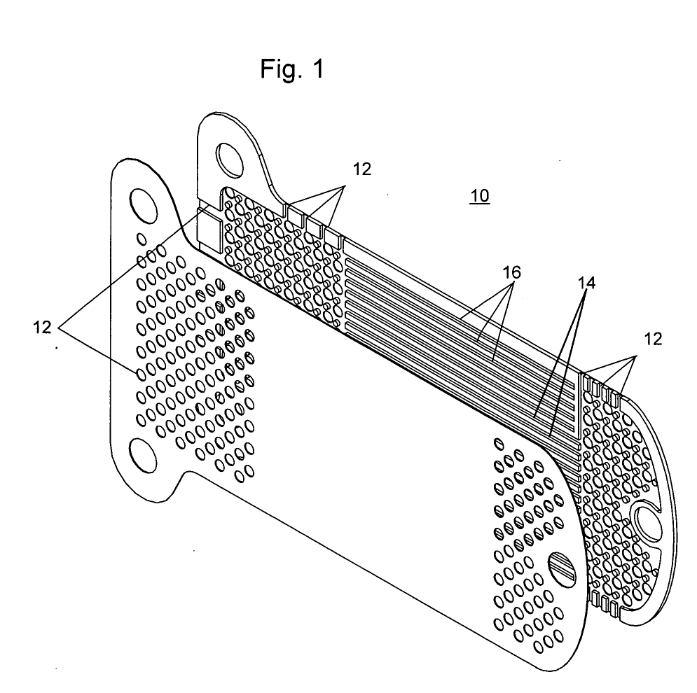

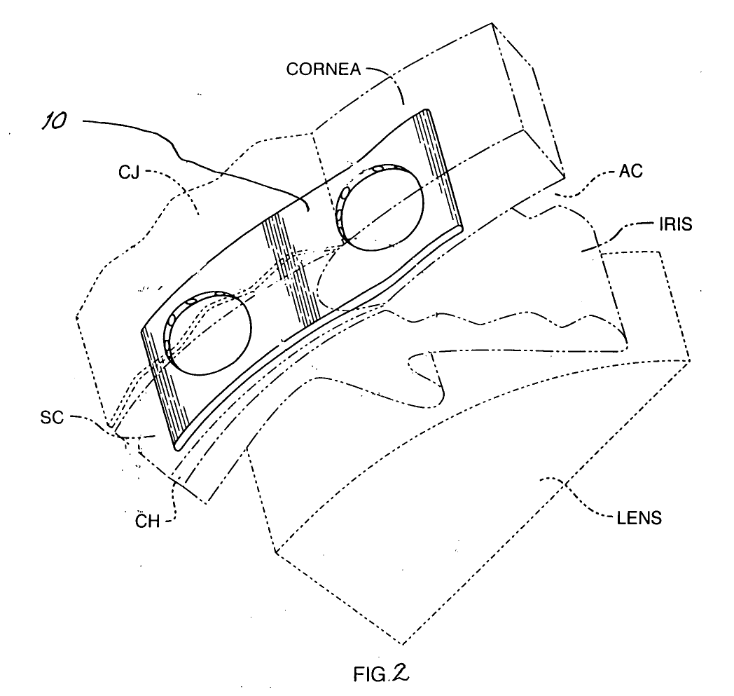

One embodiment of the present invention comprises a shunt 10 used for the treatment of glaucoma. The shunt 10, illustrated in FIG. 1, is configured from a biocompatible, non-toxic material. In one embodiment, this non-toxic biocompatible material is gold. Alloys of gold may be used, while in one embodiment 24 karat gold is used. The shunt 10 is, in one embodiment illustrated in FIG. 2, implanted in the suprachoroidal region of the eye or the supracilliary space with a first end disposed proximate to the anterior chamber. The shunt 10 comprises at least one channel through which aqueous fluid passes. Fenestrations 12 proximate to either end of the shunt 10 permit the entrance of fluid into the shunt 10 or egress of fluid from the channel 14. Apertures 12 in the shunt 10 may be opened by application of laser light of a suitable intensity and wavelength. Negative hydrostatic pressure in the supracilliary space results in a decrease in intraocular pressure when the shunt 10 introduces a pathway from the flow of intraocular fluid from the anterior chamber to the supracilliary region. The shunt may be flexible or provided with a slight curvature.

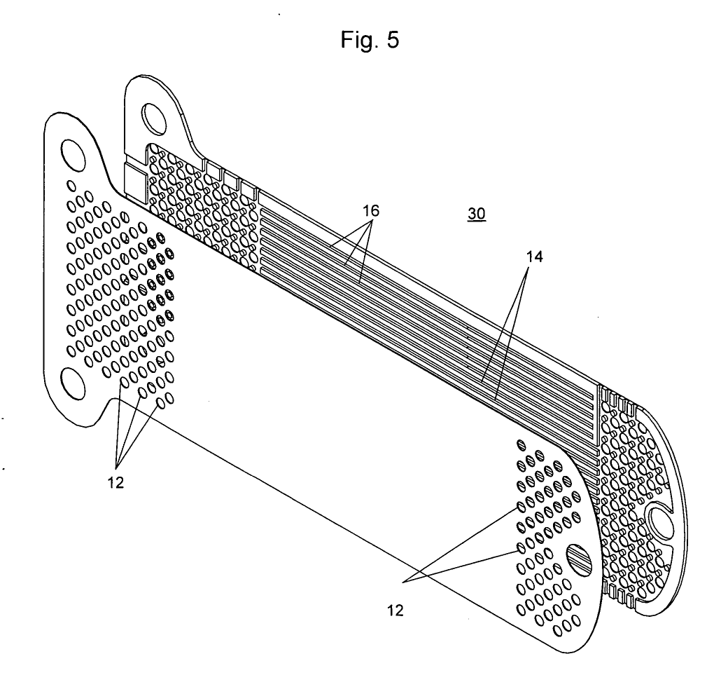

According to various embodiments, the shunt 10 is between about approximately 4 and 10 mm in length. In one such embodiment, the shunt 10 is greater than or equal to about approximately 5 mm. As illustrated in FIG. 5, the shunt 10 may be substantially longer. The shunt 10 should be of sufficient length to connect the anterior chamber with regions of the eye having a negative pressure differential. The pressure differential between the anterior chamber and the suprachoroidal region is greater, the closer to the optic nerve that one measures in the suprachoroidal region. This pressure differential has been measured as being of the order of 1 mm Hg per mm of optic means. Fluid dynamic constraints, however, dictate that the shunt 10 must not be so long as allow flow resistance to counter the benefits of the extended shunt 10.

In one embodiment, the shunt 10 may be configured with drug delivery capabilities. Drug delivery may be achieved through a variety of techniques. In some embodiments, time release coatings may be applied to the exterior or interior surfaces of the shunt 10. Alternatively, pharmaceuticals may be enclosed within the shunt 10, either within sealed channels 16 or within the walls of the shunt 10 itself. Such sealed channels 16 could then be opened through the use of a laser having an appropriate wavelength and intensity to open an aperture 12 in the shunt 10 wall. Alternatively, the shunt 10 itself could be formed using nanoscale technology to form hollows or cavities within the walls of the shunt 10. In one such embodiment, the walls of the shunt 10 may be composed of nanoshells containing pharmacological agents. The nanoshells may be fused so as to form an apparently unitary shunt 10 body. Laser ablation of the shells would, as in the case of the sealed channels or cavities 16, release the pharmaceutical into the aqueous fluid of the patient's eye.

Examples of pharmaceutical agents that may be applied to, or contained, in the shunt 10 could in some embodiments comprise beta blockers, alpha-2 antagonists, or prostaglandin analogues, such as Bimatoprost and Latanoprost. One skilled in the art will readily appreciate that the selection of pharmaceutical used would depend upon the specific needs of the patient, as some glaucoma treatments may be contraindicated for some patients having a history of other health problems or allergies while in other situations other pharmaceuticals may be found to be more efficacious to treat Glaucoma and could be introduced into the patient via the same mechanism. One skilled in the art would also readily appreciate that other diseases and syndromes may be treated through the introduction of pharmaceuticals via a shunt 10 according to one embodiment of the present invention. One skilled in the art will readily appreciate that other coatings may be applied to the shunt to facilitate the function of the shunt either through improved implantation or chemical properties of the shunt.

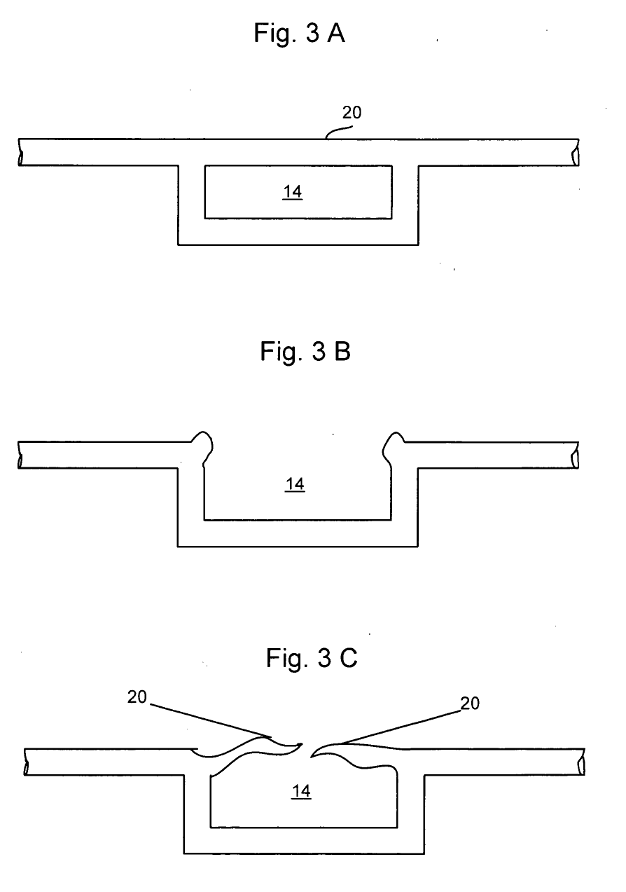

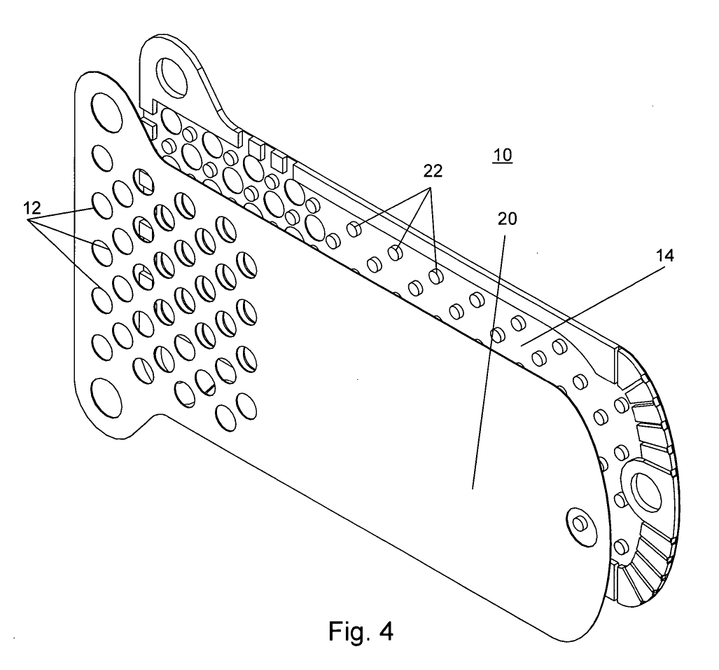

As noted above, various embodiments may be provided wherein laser light may be applied to the shunt 10 and a fenestration introduced into the shunt 10 increasing the outflow of aqueous fluid from the eye. Fenestrations 12 may be formed prior to implantation or after implantation. Post implantation fenestrations are illustrated in cross section in FIG. 3A and FIG. 3 B. Such a fenestration 12 is, according to one embodiment, achieved through the application of a titanium sapphire laser having a wavelength of between about approximately 750 nm and about approximately 800 nm and of intensity measuring. One embodiment employs a laser having a wavelength of 790 nm. The laser is directed to a laser target area 20. The number and diameter of the fenestrations 12 may be adjusted by the application of laser pulses to the wall of the shunt 10. The laser pulse melts or ablates the wall of the shunt 10, opening a fenestration 12 in the microchannel 14. Judicious application of the same laser to the periphery of a fenestration 12 thus created, results in a gradual thinning and spreading of the shunt 10 material, and a partial occlusion of the fenestration 12. Repetition of this thinning, eventually leads, as illustrated in FIG. 3C to the complete closure of the fenestration 12. Through this technique the clinician can adjust the outflow of the shunt 10 to regulate the intraocular pressure of the patient. According to such an embodiment configured such that all channels communicate with both the head and the foot of the shunt, or as illustrated in FIG. 4, a single microchannel 14 may be disposed in the shunt having a plurality of internal support structures, thereby forming a single partially obstructed microchannel. In blocked microchannels, at the head end, intakes or intake holes to these channels are occluded by a thin layer of gold, in one embodiment, approximately one fifth of the thickness of the surrounding walls of the channel. In one embodiment this layer is only 2 microns thick. A laser pulse, from a Titanium Sapphire or other laser of suitable wavelength and intensity is used to selectively ablate or melt the thin gold layer. The laser is tuned to ablate or to melt only the thickness of the layer, as the layer is significantly thinner than the surrounding shunt walls, the layer is ablated or melted without compromising the surrounding wall structure. An opening is thus created and fluid flow is permitted through the, now opened, channel. This ablation is repeated as necessary, until the desired intraocular pressure is achieved. A similar laser may be used to ablate residual tissue occluding the shunt. In one embodiment, a Titanium Sapphire laser having an intensity of between 20 and 50 mJ is used. For opening of a fenestration, higher intensity is used, while for closure of a fenestration, a lower intensity is used. In one embodiment, and intensity of 30 mJ is used to close fenestrations opened by an intensity of 50 mJ in a target 20 that is 10 microns thick.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A system for reducing intraocular pressure, the system comprising:
an implantable shunt, said implantable shunt comprising;
a substantially planar member;
at least one microchannel disposed within said planar member;
a laser whereby at least one fenestration may be introduced in to said microchannel.

2. The system according to claim 1 further comprising a closed cavity containing a pharmaceutical agent.

3. The system according to claim 2 wherein said cavity is smaller than 1 mm in diameter.

4. The system according to claim 1 wherein said laser has a wavelength between about approximately 750 nm and about approximately 800 nm.

5. The system according to claim 1 wherein a pharmaceutical agent is applied to said implantable shunt.

6. The system according to claim 1 further comprising pre implantation configured inlet and outlet apertures.

7. The system according to claim 1 wherein said laser is a titanium sapphire laser.

8. The system according to claim 1 wherein the said laser is configured to close said fenestrations when applied by a user to the edge of said fenestration.

9. A method for decreasing ocular hypertension, said method comprising:
Implanting a microchannel shunt having at least one microchannel; and
Adjusting the flow of aqueous fluid through said shunt using a laser light beam having a wavelength of between about approximately 750 and about approximately 800 nm.

10. The method according to claim 9 wherein said shunt comprises a pharmaceutical agent.

11. The method according to claim 10 wherein said agent is selected from the group of agents consisting of beta blockers, alpha-2 antagonists, and prostaglandin analogues.

12. The method according to claim 10 further comprising exposing said agent to the aqueous fluid by laser manipulation of said shunt.

13. The method according to claim 9 wherein said step of adjusting the flow of aqueous fluid through the shunt comprises introducing at least one fenestration communicating with said microchannel.

14. The method according to claim 9 wherein said step of adjusting the flow of aqueous fluid through the shunt comprises gradually closing a fenestration in said microchannel.

15. An apparatus for the treatment of ocular hypertension, said apparatus comprising:
A microchannel shunt, said shunt having a microchannel, and being configured to receive a laser pulse having a wavelength of between 750 and 800 nm;
fenestrations disposed within said microchannel, said fenestrations being configured to be opened and closed by operation of said laser.

16. The apparatus according to claim 15 wherein said shunt contains a pharmaceutical agent.

17. The apparatus according to claim 16 wherein said agent is selected from the group of agents consisting of beta blockers, alpha-2 antagonists, and prostaglandin analogues.

18. The apparatus according to claim 15 wherein said shunt is longer than 4 mm.

19. The apparatus according to claim 15 wherein said shunt is between about approximately 5 mm and about approximately 10 mm in length.

20. The apparatus according to claim 15 wherein said cavities disposed within said shunt contain pharmaceutical agents.

21. The apparatus according to claim 15 wherein said shunt comprises a single partially obstructed microchannel having structural supports disposed within the microchannel.
